SEQUENCE LISTING (12 sequences)

SEQ ID NO: 1 - protein, 302 aa, Bradyrhizobium japonicum
MCTVCGCSDGKASIEHAHDHHHDHGHDHDHGHDGHHHHHHGHDQDHHHHHDHAHGDAGLLDCGANPAGQKITGMSSDRIIQVERDILGKNDRLAADNRARFRADEVLAFNLVSSPGAGKTSLLVRAVSELKDSFAIGVIEGDQQTSNDAERIRATGVPAIQVNTGKGCHLDAAMVGEAYDRLPWLNGGLLFIENVGNLVCPAAFDLGEACKIVVFSTTEGEDKPLKYPDMFAASSLMLINKIDLASVLDFDLARTIEYARRVNPKIEVLTLSARTGEGFAAFYAWIRKRMAATTPAAMTAAE

SEQ ID NO: 2 - protein, 290 aa, Escherichia coli
MCTTCGCGEGNLYIEGDEHNPHSAFRSAPFAPAARPKMKITGIKAPEFTPSQTEEGDLHYGHGEAGTHAPGMSQRRMLEVEIDVLDKNNRLAERNRARFAARKQLVLNLVSSPGSGKTTLLTETLMRLKDSVPCAVIEGDQQTVNDAARIRATGTPAIQVNTGKGCHLDAQMIADAAPRLPLDDNGILFIENVGNLVCPASFDLGEKHKVAVLSVTEGEDKPLKYPHMFAAASLMLLNKVDLLPYLNFDVEKCIACAREVNPEIEIILISATSGEGMDQWLNWLETQRCA

SEQ ID NO: 3 - protein, 117 aa, Helicobacter pylori
MHEYSVVSSLIALCEEHAKKNQAHKIERVVVGIGERSAMDKSLFVSAFETFREESLVCKDAVLDIVDEKVELECKDCSHVFKPNALDYGVCEKCHSKNVVITQGNEMRLLSLEMLAE

SEQ ID NO: 4 - protein, 196 aa, Escherichia coli
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 5 - protein, 158 aa, Enterobacter aerogenes
MLYLTQRLEIPAAATASVTLPIDVRVKSRVKVTLNDGRDAGLLLPRGLLLRGGDVLSNEEGTEFVQVIAADEEVSVVRCDDPFMLAKACYHLGNRHVPLQIMPGELRYHHDHVLDDMLRQFGLTVTFGQLPFEPEAGAYASESHGHHHAHHDHHAHSH

SEQ ID NO: 6 - protein, 148 aa, Helicobacter pylori
MDTPNKDDSIIRFSVSLQQNLLDELDNRIIKNGYSSRSELVRDMIREKLVEDNWAEDNPNDGSKIAVLVVIYDHHQRELNQRMIDIQHTSGTHVLCTTHIHMDGHNCLETIILKGNSLEIQRLQLEIGGLRGVKFAKLTKASSFEHNE

SEQ ID NO: 7 - protein, 135 aa, Escherichia coli
MRLLHTMLRVGDLQRSIDFYTKVLGMKLLRTSENPEYKYSLAFVGYGPETEEAVIELTYNWGVDKYELGTAYGHIALSVDNAAEACEKIRQNGGNVTREAGPVKGGTTVIAFVEDPDGYKIELIEEKDAGRGLGN

SEQ ID NO: 8 - protein, 60 aa, Helicobacter pylori
MAHHEEQHGGHHHHHHHTHHHHYHGGEHHHHHHSSHHEEGCCSTSDSHHQEEGCCHGHHE

SEQ ID NO: 9 - DNA, 20 bp, Artificial Sequence
  Forward primer to T7 promoter
taatacgactcactataggg

SEQ ID NO: 10 - DNA, 19 bp, Artificial Sequence
  Reverse primer to terminator
gctagttattgctcagcgg

SEQ ID NO: 11 - protein, 5 aa, Helicobacter pylori
EEGCC

SEQ ID NO: 12 - protein, 6 aa, Artificial Sequence
  Consensus motif for nickel binding site
  Xaa (2)..(3): Can be any amino acid
CXXCGC

(19) United States
(12) Patent Application Publication
(10) Pub. No.: US 2019/0177400 A1
(43) Pub. Date: Jun. 13, 2019

(54) BIOLOGICAL UPGRADING OF HYDROCARBON STREAMS WITH NICKEL-BINDING PROTEINS

(71) Applicant: ExxonMobil Research and Engineering Company, Annandale, NJ (US)

(72) Inventors: Zarath M. Summers, High Bridge, NJ (US); Jay B. Patel, Princeton, NJ (US); Katherine G. Landuyt, Dallas, TX (US)

(21) Appl. No.: 16/186,746

(22) Filed: Nov. 12, 2018

Related U.S. Application Data
(60) Provisional application No. 62/597,488, filed on Dec. 12, 2017.

(51) Int. Cl. C07K 14/795; C12N 9/02; C12N 11/08; C10G 32/00

(52) U.S. Cl. CPC C07K 14/795; C12N 9/0069; C12N 11/08; C10G 2300/80; C10G 2300/206; C10G 2300/1077; C10G 32/00

(57) ABSTRACT

Nickel-binding proteins and methods of biologically upgrading hydrocarbon streams, such as crude oil, using nickel-binding proteins are provided herein. The nickel-binding proteins can be used to remove impurities such as metals and/or asphaltenes from a hydrocarbon stream. In some cases, the nickel-binding proteins can be chemically or genetically modified and can be used in different locations such as petroleum wells, pipes, reservoirs, tanks and/or reactors.

### CROSS-REFERENCE TO RELATED APPLICATIONS

This application claims priority to U.S. Provisional Application No. 62/597,488 filed Dec. 12, 2017 which is herein incorporated by reference in its entirety. This application is related to two other co-pending U.S. provisional applications filed on Dec. 12, 2017: U.S. Provisional Application Nos. 62/597,502 and 62/597,512, each of which is herein incorporated by reference in its entirety.

### REFERENCE TO A SEQUENCE LISTING

This application contains references to amino acid sequences and/or nucleic acid sequences which have been submitted concurrently herewith as the sequence listing text file entitled “62033524_1.txt”, file size 13 KiloBytes (KB), created on 6 Sep. 2017. The aforementioned sequence listing is hereby incorporated by reference in its entirety pursuant to 37 C.F.R. § 1.52(e)(5).

### FIELD

The present disclosure relates to nickel binding proteins for upgrading hydrocarbon streams, for example, crude oil.

### BACKGROUND

This section provides background information related to the present disclosure. The references cited in this section are not necessarily prior art.

Typically, any number of hydrocarbon streams, such as whole crude, diesel, hydrotreated oils, atmospheric gas oils, vacuum gas oils, coker gas oils, atmospheric and vacuum residues etc., may require removal of various metal species, such as vanadium and nickel, because the presence of such metals can be detrimental to refining processes. For example, metals can be particularly damaging to catalytic cracking and catalytic hydrogenation units as they can be deposited on the catalysts rendering them inactive. Nickel and vanadium, which can be abundantly found in crude oil, can be the most damaging during catalytic refining processes. However, nickel and vanadium can be very difficult to remove as they most commonly exist as oil-soluble metalloporphyrins. Chemical, thermal and physical methods have traditionally been used for metals removal. Some chemical methods include use of a demetallization agent complexation and acid treatments (sulfuric, hydrofluoric, hydrochloric). Some thermal methods include visbreaking, coking, hydrogenation and favored physical methods include distillation and solvent extraction. Unfortunately, these methods have inherent limitations. For example, chemical and thermal processing can require severe operating conditions, cause extensive side reactions, introduce product contamination, generate lower value products, and consume energy and fuel. With regard to physical methods, distillation alone can be non-selective, fail to provide complete metals removal, and solvent extraction can decrease the yield of desired hydrocarbon.

Thus, there is a need for improved methods for selectively removing impurities, such as metals. Especially needed are methods which can remove metals from hydrocarbons that leave the hydrocarbon backbone untouched, unlike some adsorption techniques. Removal of the entire hydrocarbon molecule is undesirable because up to 10 wt % of some crudes can contain heteroatoms and a 10 wt % loss of hydrocarbons is not economically feasible.

Nickel chaperones are proteins that aid in covalent folding, unfolding, assembly, or disassembly of other macromolecular structures, commonly proteins or protein complexes.

Chaperones are essential for many proteins and protein complexes to achieve their final structure. These proteins function as homo- and/or hetero-dimers. They are essential for assembling the metallocenter of nickel-containing enzymes, and sometimes contain a nickel storage domain to facilitate this process. HypA from Helicobacter pylori, HypB from Escherichia coli & Bradyrhizobium japonicum, SlyD from E. coli, and UreE from Klebsiella aerogenes are non-limiting examples of nickel chaperones.

Nickel-containing metalloenzymes are a diverse collection of enzymes. They can be simple and non-redox reactive (e.g., urease and glyoxalase I), mononuclear and redox-reactive (e.g., superoxide dismutase), heteronuclear and metal cluster containing (e.g., [nickel-iron]-hydrogenase), or nickeltetrapyrrole containing (e.g., methyl-coenzyme M reductase).

Metalloregulators are not properly considered enzymes. These proteins actively up- and down-regulate transporter expression in response to metal concentrations. The NikR family of metalloregulators is a non-limiting example of nickel regulators.

Nickel storage proteins create a non-toxic reservoir of nickel for use by the other proteins listed above. In order for metalloenzymes and regulators to be properly metallated, there must be a nontoxic intracellular supply of nickel from which chaperones can draw. Free nickel ions are highly cytotoxic, so cells produce additional storage proteins that bind free nickel. These “nickel sponges” bind nickel reversibly so they can sequester the free ions out of the cytosol and store them until they are needed by a protein for function. Hpn from H. pylori is one such protein. Hpn is very small, histidine rich, and has been over expressed in E. coli. It reversibly binds up to 5 nickel (II) ions, at short repeating motifs, per monomer with micromolar affinities. In addition to nickel (II), Hpn has also been shown to reversibly bind copper (II) and zinc (II) ions, demonstrating substrate (metal) promiscuity.

Dedeles et al. (2000) J. Biosci. & Bioengineer. 90(5):515-21 report that protoporphyrinase from Pseudomonas azelaica can degrade nickel protoporphyrin disodium (NiPPDS) to use as its sole carbon source.

Mogollon et al. (1997) Ciencia, Tecnologia y Futuro 1(3):109 report that chloroperoxidase can be used to remove 57% of total Ni & V and 23% of asphaltenes from petroporphyrin-rich crude oil fractions.

Smith et al. (2012) Prepr. Pap. Am. Chem. Soc., Div. Pet. Chem. 57(1):159 report selectively and repetitively removing up to 22% of V, Ni porphyrins from a 2:1 blend of kerosene and residue from desalted crude oil by treating with myoglobin conjugated to activated cellulose.

### SUMMARY

This section provides a general summary of the disclosure, and is not a comprehensive disclosure of its full scope or all of its features.

Nickel-binding proteins (NBPs), for example having at least 40% sequence identity to any one or more of SEQ ID NOs: 1-8 to upgrade the quality of hydrocarbon streams, are disclosed herein. In certain embodiments, an NBP comprises a nickel chaperone, for example a chaperone having at least 40% sequence identity to any one or more of SEQ ID NOs: 1-5. In certain embodiments, an NBP comprise a metalloenzyme, for example a metalloenzyme having at least 40% sequence identity to SEQ ID NO:6. In certain embodiments, an NBP comprises a metalloregulator, for example a metalloregulator having at least 40% sequence identity to SEQ ID NO:7. In certain embodiments, an NBP comprises a nickel storage protein, for example a storage protein having at least 40% sequence identity to SEQ ID NO:8. Compositions comprising an NBP for upgrading hydrocarbon streams are also provided herein.

Also disclosed herein are recombinant or modified NBPs, in which the NBP has been made more hydrophobic than its native counterpart. In certain embodiments, the NBP is hydrophobically modified to be at least 10% more enriched in hydrophobic amino acids selected from the group consisting of Ala, Gly, Ile, Leu, Met, Pro, Phe, and Trp. In certain embodiments, additional hydrophobic amino acids are added to the NBP. In certain embodiments, amino acids with polar or charged side chains are replaced with hydrophobic amino acids. In certain embodiments the NBP is treated chemically (e.g., NBP is rinsed with n-propanol, NBP is conjugated to a polyethylene glycol, or disulfide bridges are added to the NBP) to be more hydrophobic.

Methods of biologically upgrading hydrocarbon streams, such as crude oil, are additionally disclosed herein. These methods involve contacting the hydrocarbon stream with the NBPs and/or compositions described herein. In certain embodiments, the contacting occurs while the hydrocarbon streams are moved through pipes or stored in reservoirs or tanks. In certain embodiments, the contacting occurs while the hydrocarbon streams are present in a reactor. In certain embodiments, the contacting occurs before the hydrocarbon stream, e.g., crude oil, may be extracted from the earth, for example by sending the NBPs and/or compositions described herein into a petroleum well. In certain embodiments, the contacting results in the removal of impurities (e.g., metal or asphaltenes) from the hydrocarbon stream.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments, and not all possible implementations. The drawings and their corresponding descriptions are not intended to limit the scope of the present disclosure.

FIG. 1 shows UV-VIS absorbance spectra for: (1A) Ni(II)-protoporphyrin (IX); (1B) Ni(II)-meso-tetra-(4-carboxyphenyl) porphine; (1C) Ni(II)-meso-tetra-(N-methyl-4-pyridyl) porphine tetrachloride; and (1D) VO-meso-tetra-(N-methyl-4-pyridyl) porphine tetrachloride upon treatment with crude and purified B. japonicum HypB. HypB treated samples are shown at 0 hr (solid gray line) and 24 hr (dashed gray line). pET28b empty vector crude protein lysate treated samples are shown at 0 hr (solid black line) and 24 hr (dashed black line).4 shows the percentage of initial fluorene that is converted into more refined product by the various E. coli strains indicated.

FIG. 2 shows UV-VIS absorbance spectra for: (2A) Ni(II)-protoporphyrin (IX); and (2B) Ni(II)-meso-tetra-(4-carboxyphenyl) porphine upon treatment with crude and purified E. coli HypB. HypB treated samples are shown at 0 hr (solid gray line) and 24 hr (dashed gray line). pET28b empty vector crude protein lysate treated samples are shown at 0 hr (solid black line) and 24 hr (dashed black line).

FIG. 3 shows UV-VIS absorbance spectra for: (3A) Ni(II)-protoporphyrin (IX); and (3B) Ni(II)-meso-tetra-(4-carboxyphenyl) porphine upon treatment with crude and purified HypA. HypA treated samples are shown at 0 hr (solid gray line) and 24 hr (dashed gray line). pET28b empty vector crude protein lysate treated samples are shown at 0 hr (solid black line) and 24 hr (dashed black line).

FIG. 4 shows UV-VIS absorbance spectra for: (4A) Ni(II)-protoporphyrin (IX); and (4B) Ni(II)-meso-tetra-(4-carboxyphenyl) porphine upon treatment with crude and purified SlyD. SlyD treated samples are shown at 0 hr (solid gray line) and 24 hr (dashed gray line). pET28b empty vector crude protein lysate treated samples are shown at 0 hr (solid black line) and 24 hr (dashed black line).

FIG. 5 shows UV-VIS absorbance spectra for: (5A) Ni(II)-protoporphyrin (IX); and (5B) Ni(II)-meso-tetra-(4-carboxyphenyl) porphine upon treatment with crude and purified UreE. UreE treated samples are shown at 0 hr (solid gray line) and 24 hr (dashed gray line). pET28b empty vector crude protein lysate treated samples are shown at 0 hr (solid black line) and 24 hr (dashed black line).

FIG. 6 shows UV-VIS absorbance spectra for: (6A) Ni(II)-protoporphyrin (IX); and (6B) Ni(II)-meso-tetra-(4-carboxyphenyl) porphine upon treatment with crude and purified NikR. NikR treated samples are shown at 0 hr (solid gray line) and 24 hr (dashed gray line). pET28b empty vector crude protein lysate treated samples are shown at 0 hr (solid black line) and 24 hr (dashed black line).

FIG. 7 shows UV-VIS absorbance spectra for: (7A) Ni(II)-protoporphyrin (IX); and (7B) Ni(II)-meso-tetra-(4-carboxyphenyl) porphine upon treatment with crude and purified GloA. GloA treated samples are shown at 0 hr (solid gray line) and 24 hr (dashed gray line). pET28b empty vector crude protein lysate treated samples are shown at 0 hr (solid black line) and 24 hr (dashed black line).

FIG. 8 shows UV-VIS absorbance spectra for: (8A) Ni(II)-protoporphyrin (IX); and (8B) Ni(II)-meso-tetra-(4-carboxyphenyl) porphine upon treatment with crude Hpn lysates. Hpn treated samples are shown at 0 hr (solid colored line) and 24 hr (dashed colored line). pET28b empty vector crude protein lysate treated samples are shown at 0 hr (solid black line) and 24 hr (dashed black line).

### DETAILED DESCRIPTION

Unless defined otherwise, all technical and scientific terms used herein have the same meaning as commonly understood by one of ordinary skill in the art to which this invention belongs. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

In case of conflict between definitions incorporated by reference and definitions set out in the present disclosure, the definitions of the present disclosure will control.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

### I. Definitions

To facilitate an understanding of the present invention, a number of terms and phrases are defined below.

As used in the present disclosure and claims, the singular forms “a,” “an,” and “the” include plural forms unless the context clearly dictates otherwise.

Wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include “A and B,” “A or B,” “A,” and “B.”

As used herein, and unless otherwise specified, the term “Cn” means hydrocarbon(s) having n carbon atom(s) per molecule, wherein n is a positive integer.

As used herein, the term “hydrocarbon(s)” means a class of compounds containing hydrogen bound to carbon, which may be linear, branched or cyclic, and encompasses (i) saturated hydrocarbon compounds, (ii) unsaturated hydrocarbon compounds, and (iii) mixtures of hydrocarbon compounds (saturated and/or unsaturated) including mixtures of hydrocarbon compounds having different values of n. The term “hydrocarbon(s)” is also intended to encompass hydrocarbons containing one or more heteroatoms, such as, but not limited to nitrogen, sulfur, and oxygen, and/or containing one or more metals, such as vanadium and nickel. Non-limiting examples of heteroatom-containing and metal-containing hydrocarbons include porphyrins or petroporphyrins, and metalloporphyrins. The term “porphyrin” refers to a cyclic structure typically composed of four modified pyrrole rings interconnected at their a carbon atoms via methane bridges (=C—) and having two replaceable hydrogens on two nitrogens, where, for example, various metal atoms can be substituted to form a metalloporphyrin. Examples of nitrogen-containing species include, but are not limited to carbazoles, imidazoles, pyrroles, quinones, quinilines and combinations thereof. Examples of sulfur-containing species include, but are not limited to mercaptans, thiols, disulfides, thiophenes, benzothiophenes, dibenzothiophenes and combinations thereof. Examples of oxygen-containing species include, but are not limited to furans, indoles, carbazoles, benzcarbazoles, pyridines, quinolines, phenanthridines, hydroxypyridines, hydroxyquinolines, dibenzofuranes, naphthobenzofuranes, phenols, aliphatic ketones, carboxylic acids, and sulfoxides.

As used herein, the term “hydrocarbon stream” refers to any stream comprising hydrocarbons, which may be present in the oil reservoir/wellbore, pipes, tanks, reactors, etc. Examples of hydrocarbon streams include, but are not limited to hydrocarbon fluids, whole crude oil, diesel, kerosene, virgin diesel, light gas oil (LGO), lubricating oil feedstreams, heavy coker gasoil (HKGO), de-asphalted oil (DAO), fluid catalytic cracking (FCC) main column bottom (MCB), steam cracker tar, streams derived from crude oils, shale oils and tar sands, streams derived from the Fischer-Tropsch processes, reduced crudes, hydrocrackates, raffinates, hydrotreated oils, atmospheric gas oils, vacuum gas oils, coker gas oils, atmospheric and vacuum residues (vacuum resid), deasphalted oils, slack waxes and Fischer-Tropsch wax. The hydrocarbon streams may be derived from various refinery units, such as, but not limited to distillation towers (atmospheric and vacuum), hydrocrackers, hydrotreaters and solvent extraction units.

As used herein, the term “asphaltene” refers to a class of hydrocarbons, present in various hydrocarbon streams, such as crude oil, bitumen, or coal, that are soluble in toluene, xylene, and benzene, yet insoluble in paraffinic solvents, such as n-alkanes, e.g., n-heptane and n-pentane. Asphaltenes may be generally characterized by fused ring aromaticity with some small aliphatic side chains, and typically some polar heteroatom-containing functional groups, e.g., carboxylic acids, carbonyl, phenol, pyrroles, and pyridines, capable of donating or accepting protons intermolecularly and/or intramolecularly. Asphaltenes may be characterized as a high molecular weight fraction of crude oils, e.g., an average molecular weight (about 1000 and up to 5,000) and very broad molecular weight distribution (up to 10,000), and high coking tendency.

As used herein, the term “upgrade” or “upgrading” generally means to improve quality and/or properties of a hydrocarbon stream and is meant to include physical and/or chemical changes to a hydrocarbon stream. Further, upgrading is intended to encompass removing impurities (e.g., heteroatoms, metals, asphaltenes, etc.) from a hydrocarbon stream, converting a portion of the hydrocarbons into shorter chain length hydrocarbons, cleaving single ring or multi-ring aromatic compounds present in a hydrocarbon stream, and/or reducing viscosity of a hydrocarbon stream.

As used herein, the term “hydrophobic” refers to a substance or a moiety, which lacks an affinity for water. That is, a hydrophobic substance or moiety tends to substantially repel water, is substantially insoluble in water, does not substantially mix with or be wetted by water or to do so only to a very limited degree and/or does not absorb water or, again, to do so only to a very limited degree.

The term “heterologous” with regard to a gene regulatory sequence (such as, for example, a promoter) means that the regulatory sequence or is from a different source than the nucleic acid sequence (e.g., protein coding sequence) with which it is juxtaposed in a nucleic acid construct. By way of non-limiting example, a slyD gene from E. coli is heterologous to a slyD promoter from Y. pestis. Similarly, the slyD gene is heterologous to the hypB promoter, even when both slyD and hypB are from E. coli.

The term “expression cassette,” as used herein, refers to a nucleic acid construct that encodes a protein or functional RNA (e.g. a tRNA, a short hairpin RNA, one or more microRNAs, a ribosomal RNA, etc.) operably linked to expression control elements, such as a promoter, and optionally, any or a combination of other nucleic acid sequences that affect the transcription or translation of the gene, such as, but not limited to, a transcriptional terminator, a ribosome binding site, a splice site or splicing recognition sequence, an intron, an enhancer, a polyadenylation signal, an internal ribosome entry site, etc.

The term “operably linked,” as used herein, denotes a configuration in which a control sequence is placed at an appropriate position relative to the coding sequence of a polynucleotide sequence such that the control sequence directs the expression of the coding sequence of a polypeptide and/or functional RNA). Thus, a promoter is in operable linkage with a nucleic acid sequence if it can mediate transcription of the nucleic acid sequence. When introduced into a host cell, an expression cassette can result in transcription and/or translation of an encoded RNA or polypeptide under appropriate conditions. Antisense or sense constructs that are not or cannot be translated are not excluded by this definition. In the case of both expression of transgenes and suppression of endogenous genes (e.g., by antisense, or sense suppression) one of ordinary skill will recognize that the inserted polynucleotide sequence need not be identical, but may be only substantially identical to a sequence of the gene from which it was derived. As explained herein, these substantially identical variants are specifically covered by reference to a specific nucleic acid sequence.

“Naturally-occurring” and “wild-type” (WT) refer to a form found in nature. For example, a naturally occurring or wild-type nucleic acid molecule, nucleotide sequence, or protein may be present in, and isolated from, a natural source, and is not intentionally modified by human manipulation.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window. The degree of amino acid or nucleic acid sequence identity can be determined by various computer programs for aligning the sequences to be compared based on designated program parameters. For example, sequences can be aligned and compared using the local homology algorithm of Smith & Waterman (1981) Adv. Appl. Math. 2:482-89, the homology alignment algorithm of Needleman & Wunsch (1970) J. Mol. Biol. 48:443-53, or the search for similarity method of Pearson & Lipman (1988) Proc. Nat'l. Acad. Sci. USA 85:2444-48, and can be aligned and compared based on visual inspection or can use computer programs for the analysis (for example, GAP, BESTFIT, FASTA, and TFASTA in the Wisconsin Genetics Software Package, Genetics Computer Group, 575 Science Dr., Madison, Wis.).

The BLAST algorithm, described in Altschul et al. (1990) J. Mol. Biol. 215:403-10, is publicly available through software provided by the National Center for Biotechnology Information (at the web address www.ncbi.nlm.nih.gov). This algorithm identifies high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). Initial neighborhood word hits act as seeds for initiating searches to find longer HSPs containing them. The word hits are then extended in both directions along each sequence for as far as the cumulative alignment score can be increased. Cumulative scores are calculated for nucleotides sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. For determining the percent identity of an amino acid sequence or nucleic acid sequence, the default parameters of the BLAST programs can be used. For analysis of amino acid sequences, the BLASTP defaults are: word length (W), 3; expectation (E), 10; and the BLOSUM62 scoring matrix. For analysis of nucleic acid sequences, the BLASTN program defaults are word length (W), 11; expectation (E), 10; M=5; N=−4; and a comparison of both strands. The TBLASTN program (using a protein sequence to query nucleotide sequence databases) uses as defaults a word length (W) of 3, an expectation (E) of 10, and a BLOSUM 62 scoring matrix. See, Henikoff & Henikoff (1992) Proc. Nat'l. Acad. Sci. USA 89:10915-19.

In addition to calculating percent sequence identity, the BLAST algorithm also performs a statistical analysis of the similarity between two sequences (see, e.g., Karlin & Altschul (1993) Proc. Nat'l. Acad. Sci. USA 90:5873-87). The smallest sum probability (P(N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, preferably less than about 0.01, and more preferably less than about 0.001.

“Pfam” is a large collection of protein domains and protein families maintained by the Pfam Consortium and available at several sponsored World Wide Web sites. Pfam domains and families are identified using multiple sequence alignments and hidden Markov models (HMMs). Pfam-A families, which are based on high quality assignments, are generated by a curated seed alignment using representative members of a protein family and profile hidden Markov models based on the seed alignment, whereas Pfam-B families are generated automatically from the non-redundant clusters of the latest release of the Automated Domain Decomposition algorithm (ADDA; Heger A, Holm L (2003) J Mol Biol 328(3):749-67). All identified sequences belonging to the family are then used to automatically generate a full alignment for the family (Sonnhammer et al. (1998) Nucleic Acids Research 26: 320-322; Bateman et al. (2000) Nucleic Acids Research 26: 263-266; Bateman et al. (2004) Nucleic Acids Research 32, Database Issue: D138-D141; Finn et al. (2006) Nucleic Acids Research Database Issue 34: D247-251; Finn et al. (2010) Nucleic Acids Research Database Issue 38: D211-222).

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. et al., (1979) Principles of Protein Structure, Springer-Verlag). According to such analyses, groups of amino acids can be defined where amino acids within a group exchange preferentially with each other, and therefore resemble each other most in their impact on the overall protein structure (Schulz, G. E. et al., (1979) Principles of Protein Structure, Springer-Verlag). Examples of amino acid groups defined in this manner include an “aromatic or cyclic group,” including Pro, Phe, Tyr, and Trp. Within each group, subgroups can also be identified. For example, the group of charged amino acids can be sub-divided into sub-groups including: the “positively-charged sub-group,” comprising Lys, Arg and His; and the “negatively-charged sub-group,” comprising Glu and Asp. In another example, the aromatic or cyclic group can be sub-divided into sub-groups including: the “nitrogen ring sub-group,” comprising Pro, His, and Trp; and the “phenyl sub-group” comprising Phe and Tyr. In another further example, the hydrophobic group can be sub-divided into sub-groups including: the “large aliphatic non-polar sub-group,” comprising Val, Leu, and Ile; the “aliphatic slightly-polar sub-group,” comprising Met, Ser, Thr, and Cys; and the “small-residue sub-group,” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn such that a free —NH₂ can be maintained.

### II. Nickel-Binding Proteins

As disclosed herein, the present inventors have discovered that various classes of NBPs can be used to upgrade hydrocarbon streams. By contacting a hydrocarbon stream (e.g., crude oil) with the NBP, impurities such as metals and asphaltenes can be removed and properties of the hydrocarbon stream can be improved, for example, viscosity may be lowered. Additionally, the fraction of the upgraded product that is recoverable can be increased.

In certain embodiments, the NBP can be an NBP that classifies as belonging to any one or more of Pfam families PF00254, PF01155, PF02492, and PF05194. In certain embodiments, the NBP classifies as belonging to Pfam family PF00903. In certain embodiments, the NBP classifies to Pfam family PF08753 and/or PF01402. In certain embodiments, the NBP comprises two or more repeats of the sequence EEGCC (SEQ ID NO:11) in a 50 amino acid stretch. Although the NBP(s) can be present in the context of a host cell (e.g., a microbial cell), in certain embodiments the NBPs are substantially free or even totally free of cells, cell components, or cellular debris beyond the bare NBP itself

In certain embodiments, the NBP has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:1.

In certain embodiments, the NBP has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:2.

In certain embodiments, the NBP has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:3.

In certain embodiments, the NBP has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:4

In certain embodiments, the NBP has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:5.

In certain embodiments, the NBP has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:6.

In certain embodiments, the NBP has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:7.

In certain embodiments, the NBP has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:8.

A. Hydrophobic Modification

In certain embodiments, the NBPs described herein can be modified to become more hydrophobic. Because the hydrocarbon stream may be a hydrophobic environment, by making the NBP (in particular those NBP surfaces that are exposed to the hydrophobic environment of the hydrocarbon stream) more hydrophobic, the NBP can be better able to tolerate the stresses of the environment.

In certain embodiments, the NBPs can be modified to be more hydrophobic by the inclusion of a greater number of hydrophobic amino acids (Ala, Gly, Ile, Leu, Met, Pro, Phe, and Trp) in the NBP's primary sequence. This can be accomplished in a number of different ways, none of which are mutually exclusive of each other. For example, one can replace a given polar (Asn, Cys, Gln, Ser, Thr, and Tyr) or charged (Arg, Asp, Glu, His, and Lys) with a hydrophobic amino acid. Additionally or alternatively, one can add one or more additional hydrophobic amino acid between two amino acids already present in the primary sequence of the wild type. Additionally or alternatively, one can add one or more (e.g., at least 5, at least 10, at least 20, at least 30, at least 40, or at least 50) additional hydrophobic amino acids at the amino and/or carboxy terminus of the NBP. The result of these additions and/or substitutions can result in an NBP that is at least 5% (e.g., at least 10%, at least 15%, at least 20%, at least 25%, at least 30%, at least 35%, at least 40%, at least 45%, or at least 50%) more hydrophobic than the corresponding wild-type NBP sequence.

In order for an NBP's amino acid sequence to be modified relative to the corresponding wild type sequence, the modified sequence must be less than 100% identical to its corresponding wild type sequence. In certain embodiments, the modified NBP is no more than about 95% identical to the corresponding wild type, for example no more than about 90%, no more than about 85%, no more than about 80%, no more than about 75%, no more than about 70%, no more than about 65%, or no more than about 70% identical. However, the modified NBP will still be at least about 40% (for example, at least 50%, at least 60%, at least 70%, at least 80%, at least 90%, at least 91%, at least 92%, at least 93%, or at least 94%) identical to the corresponding wild type sequence (e.g., a sequence selected from the group consisting of SEQ ID NOs:1-8).

Additionally or alternatively, in certain embodiments an NBP can be made more hydrophobic by chemical modification. In certain embodiments, the NBP can be rinsed with n-propanol. In certain embodiments polyethylene glycol can be conjugated to the NBP. In certain embodiments, disulfide bridges can be added to the NBP. The addition of disulfide bridges can affect the NBP's tertiary structure. Therefore additional disulfide bridges must be placed carefully. The person of ordinary skill knows how to place disulfide bridges in a manner that will cause minimal disruption to nickel binding.

B. Nucleic Acids

Also described herein are nucleic acids encoding NBPs for use with the methods and compositions described herein. The person of ordinary skill knows that the degeneracy of the genetic code permits a great deal of variation among nucleotides that all encode the same protein. For this reason, it is to be understood that the representative nucleotide sequences disclosed herein are not intended to limit the understanding of phrases such as “a nucleotide encoding a protein having at least 70% identity to SEQ ID NO . . . ” or “a construct encoding SEQ ID NO . . . ”.

In certain embodiments, the nucleotide encodes an NBP having at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) identity to a sequence selected from the group consisting of SEQ ID NOs:1-8. In certain embodiments, the nucleotide encodes a protein that is hydrophobically modified as described herein. In order for an NBP's amino acid sequence to be modified relative to the corresponding wild type sequence, the modified sequence must be less than 100% identical to its corresponding wild type sequence. In certain embodiments, the nucleotide encodes a modified NBP that is no more than about 95% identical to the corresponding wild type, for example no more than about 90%, no more than about 85%, no more than about 80%, no more than about 75%, no more than about 70%, no more than about 65%, or no more than about 60% identical. However, the modified NBP will still be at least about 40% (for example, at least 50%, at least 60%, at least 70%, at least 80%, at least 90%, at least 91%, at least 92%, at least 93%, or at least 94%) identical to the corresponding wild type sequence (e.g., a sequence selected from the group consisting of SEQ ID NOs: 1-8).

In certain embodiments, the nucleotides described herein are incorporated into expression cassettes. The choice of regulator elements such as promoter or terminator or splice site for use in expression cassettes depends on the intended cellular host for gene expression. The person of ordinary skill knows how to select regulatory elements appropriate for an intended cellular host. A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in both directions off of opposite strands). A promoter may be a constitutive promoter, a repressible promoter, or an inducible promoter. Non-limiting examples of promoters include, for example, the T7 promoter, the cytomegalovirus (CMV) promoter, the SV40 promoter, and the RSV promoter. Examples of inducible promoters include the lac promoter, the pBAD (araA) promoter, the Tet promoter (U.S. Pat. Nos. 5,464,758 and 5,814,618), and the Ecdysone promoter (No et al. (1996) Proc. Natl. Acad. Sci. 93:3346-51).

In certain embodiments, the nucleotides and/or expression cassettes described herein can be incorporated into vectors. A vector can be a nucleic acid that has been generated via human intervention, including by recombinant means and/or direct chemical synthesis, and can include, for example, more than one or more of: 1) an origin of replication for propagation of the nucleic acid sequences in one or more hosts (which may or may not include the production host); 2) one or more selectable markers; 3) one or more reporter genes; 4) one or more expression control sequences, such as, but not limited to, promoter sequences, enhancer sequences, terminator sequences, sequence for enhancing translation, etc.; and/or 5) one or more sequences for promoting integration of the nucleic acid sequences into a host genome, for example, one or more sequences having homology with one or more nucleotide sequences of the host microorganism. A vector can be an expression vector that includes one or more specified nucleic acid “expression control elements” that permit transcription and/or translation of a particular nucleic acid in a host cell. The vector can be a plasmid, a part of a plasmid, a viral construct, a nucleic acid fragment, or the like, or a combination thereof. In certain embodiments, the expression cassettes or vectors described herein can comprise genes encoding the hydrophobically modified NBPs described above.

In certain embodiments the nucleotide coding sequences may be revised to produce messenger RNA (mRNA) with codons preferentially used by the host cell to be transformed (“codon optimization”). Thus, for enhanced expression of transgenes, the codon usage of the transgene can be matched with the specific codon bias of the organism in which the transgene is desired to be expressed. The precise mechanisms underlying this effect are believed to be many, but can include the proper balancing of available aminoacylated tRNA pools with proteins being synthesized in the cell, coupled with more efficient translation of the transgenic mRNA when this need is met. In some examples, only a portion of the codons is changed to reflect a preferred codon usage of a host microorganism. In certain examples, one or more codons are changed to codons that are not necessarily the most preferred codon of the host microorganism encoding a particular amino acid. Additional information for codon optimization is available, e.g. at the codon usage database of GenBank. The coding sequences may be codon optimized for optimal production of a desired product in the host organism selected for expression. In certain examples, the nucleic acid sequence encoding an NBP is codon optimized for expression in E. coli. In some aspects, the nucleic acid molecules of the invention encode fusion proteins that comprise an NBP. For example, the nucleic acids of the invention may comprise polynucleotide sequences that encode glutathione-S-transferase (GST) or a portion thereof, thioredoxin or a portion thereof, maltose binding protein or a portion thereof, poly-histidine (e.g. His6), poly-HN, poly-lysine, a hemagglutinin tag sequence, HSV-Tag, and/or at least a portion of HIV-Tat fused to the NBP-encoding sequence.

The vector can be a high copy number vector, a shuttle vector that can replicate in more than one species of cell, an expression vector, an integration vector, or a combination thereof. Typically, the expression vector can include a nucleic acid comprising a gene of interest operably linked to a promoter in an expression cassette, which can also include, but is not limited to, a localization peptide encoding sequence, a transcriptional terminator, a ribosome binding site, a splice site or splicing recognition sequence, an intron, an enhancer, a polyadenylation signal, an internal ribosome entry site, and similar elements.

C. Expression in Host Cells

In a further aspect, a recombinant microorganism or host cell, such as a recombinant E. coli comprising a non-native gene encoding an NBP (e.g., a hydrophobically modified NBP) is disclosed herein. In certain embodiments, the NBP comprises an amino acid sequence having at least about 40% sequence identity to a sequence selected from the group consisting of SEQ ID NOs:1-8, and/or to an active fragment of any thereof. For example, the non-native gene can encode an NBP having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a sequence selected from the group consisting of SEQ ID NOs:1-8. In certain embodiments, the sequence having at least about 40% identity to a sequence selected from the group consisting of SEQ ID NOs:1-8 is modified as described herein to make the resulting protein more hydrophobic than its wild-type counterpart.

In certain embodiments, the host cell can be a prokaryotic host cell, either gram negative or gram positive. By way of non-limiting example, the host cell can be an E. coli host cell. The skilled artisan is familiar with the media and techniques necessary for the culture of prokaryotic host cells, including E. coli.

In certain embodiments, the host cell can be a eukaryotic host cell, such as a yeast (e.g., S. cerevisiae or S. pombe) or an insect cell (e.g., an Spodoptera frugiperda cell such as Sf9 or Sf21). The skilled artisan is familiar with the media and techniques necessary for the culture of eukaryotic host cells, including yeast and insect cells.

### III. Compositions/Combinations

The NBPs described herein can be used in particular circumstances (e.g., bore holes, fuel tanks, pipes, reactor surfaces, etc.) common to the petroleum industry. In such circumstances, it may be useful to combine the NBPs with other relevant ingredients (including other NBPs) appropriate to the use intended or the environment into which the NBPs are being placed. The mixture of various NBPs creates “combinations.” The addition of other, non-NBP, ingredients to one or more NBPs creates a “composition.”

In certain embodiments, combinations described herein may comprise: both a nickel chaperone and a metalloenzyme; or both of a nickel chaperone and a metalloregulator; or both of a nickel chaperone and a nickel storage protein; or both a metalloenzyme and a metalloregulator; or both a metalloenzyme and a nickel storage protein; or both a metalloregulator and nickel storage protein; or all three of a nickel chaperone, a metalloenzyme, and a metalloregulator; or all three of a nickel chaperone, a metalloenzyme, and a nickel storage protein; or all three of a nickel chaperone, a metalloregulator, and a nickel storage protein; or all three of a metalloenzyme, a metalloregulator, and a nickel storage protein; or all four of a nickel chaperon, a metalloenzyme, a metalloregulator, and a nickel storage protein.

Additionally or alternatively, the compositions described herein can comprise one or more NBP and one or more enzyme useful for upgrading hydrocarbon streams, such as oxygenases and/or dioxygenases enzymes. These enzymes can be useful for removing impurities (e.g. metals, heteroatoms and/or asphaltenes).

Additionally or alternatively, the compositions described herein can comprise one or more NBP along with one or more of a lubricant, a surfactant, a viscosity additive, a fluid loss additive, a foam control agent, a weighting material, and a salt.

### IV. Methods of Use

Also provided herein are methods of using the NBPs and compositions described herein. In various aspects, methods of biologically upgrading a hydrocarbon stream are provided herein comprising contacting a reservoir of less refined fuel (e.g., crude oil or vacuum resid) with an NBP or composition as described herein to reduce impurities in the fuel. In certain embodiments, the impurity can be a metal, for example transition metals, such as, but not limited to nickel, vanadium, zinc, copper, cobalt, cadmium, chromium, particularly, nickel and vanadium. In certain embodiments, the impurity can be an asphaltene. In certain embodiments, the hydrocarbon stream can comprise multiple impurities, and the methods disclosed herein can be effective against more than one impurity, and sometimes against all impurities in the fuel.

In some embodiments, the NBPs described herein may be capable of flexing or opening a metal-containing compound, e.g., metal-containing porphyrin compounds, in a hydrocarbon stream such that the metal (e.g., Ni, V) may be released or removed from the hydrocarbon stream while the hydrocarbon from which it is released (e.g., porphyrin) substantially remains. It also contemplated herein, that the NBPs described herein also may be capable of selectively removing a metal-containing compound, e.g., metal-containing porphyrin compounds, in its entirety from a hydrocarbon stream.

In certain embodiments, the hydrocarbon stream is contacted with an NBP described herein. In some embodiments, the upgrading can comprise removing at least a portion of impurities from the hydrocarbon stream. Exemplary impurities include, but are not limited to metals (e.g., nickel and/or vanadium), asphaltenes, and combinations thereof. In certain embodiments, the methods disclosed herein can enhance recovery at least 10% (for example, at least 15%, at least 20%, at least 25%, at least 30%, at least 35%, at least 40%, at least 45%, or at least 50%) relative to the amount of refined fuel recovered from the same quantum of hydrocarbon feed, but without the step of contacting with the NBP.

In other embodiments, the upgrading methods described herein can enhance the quantity of hydrocarbons recovered from a hydrocarbon stream or limit the loss of hydrocarbons, for example, the NBP described herein can selectively remove impurities from hydrocarbon compounds in the hydrocarbon stream without removing the entire hydrocarbon molecules, i.e., leaving the hydrocarbon backbone substantially untouched. Thus, in some embodiments, there can be lower loss of hydrocarbons following separation of the impurities from the hydrocarbon stream, for example, a loss of ≤15 wt %, ≤10 wt %, ≤8.0 wt %, ≤5.0 wt %, or ≤1.0 wt % of hydrocarbons may occur after separation of the impurities from the hydrocarbon stream.

The hydrocarbon stream may contacted with the NBP and compositions described herein for any suitable amount of time. Advantageously, upgrading of the hydrocarbon stream when contacted with the NBP described herein may occur in a short period of time, for example, the hydrocarbon stream may be contacted with dioxygenases for ≤about 10 hours, ≤about 5.0 hours, ≤about 1.0 hours, ≤about 30 minutes, ≤about 10 minutes, ≤about 1.0 minutes, ≤about 30 seconds, ≤about 10 seconds or ≤about 1.0 second.

Advantageously, the methods described here can be performed across a wide range of pressures and temperatures and even at ambient pressure and temperature. Effective upgrading conditions can include temperatures of about 15° C. to about 30° C. and pressures of from about 90 kPa to about 200 kPa. Additionally or alternatively, upgrading can be performed at higher temperatures of about 30° C. to about 200° C. or 30° C. to about 120° C.

### V. Locations, Forms and Immobilization

The methods described herein can be performed in various locations. For example, the NBPs may be present in an oil reservoir/wellbore, a pipeline, a tank, a vessel, a reactor, a waste water stream (e.g., exiting a reactor), a waste water pond, or any combinations thereof. In a particular embodiment, an NBP may contact crude oil in the oil reservoir/wellbore, for example, through injection into the oil reservoir/wellbore. In another particular embodiment, an NBP may contact a hydrocarbon stream, e.g., crude oil or hydrocarbon product stream, as it flows and/or resides in a pipeline and/or a holding vessel or a tank. When added to a pipeline and/or a holding vessel or a tank, a hydrocarbon stream may be upgraded without any substantially additional processing time, for example, when a hydrocarbon stream is awaiting further processing and/or transport.

In certain embodiments, the NBPs and compositions described herein can be present in free form or crystal form, while in other embodiments the NBPs and compositions can be immobilized on a carrier or scaffold, such as a membrane, a filter, a matrix, diatomaceous material, particles, beads, in an ionic liquid coating, an electrode, or a mesh. In certain embodiments, the NBP may be thermally stable from about 15° C. to about 150° C., about 50° C. to about 120° C. or about 90° C. to about 120° C.

In certain embodiments, the NBPs and compositions described herein can be present in crystal form and the crystals can be added to hydrocarbon streams at the various locations listed above. Standard techniques known to a person of ordinary skill in the art may be used to form to NPB crystals.

Additionally or alternatively, the NPBs and compositions described herein can be immobilized by standard techniques known to a person of ordinary skill in the art, and the hydrocarbon stream may contact an immobilized NBP by flowing over, through, and/or around the immobilized NBP. Suitable carriers or scaffolds include, but are not limited to a membrane, a filter, a matrix, diatomaceous material, particles, beads, an ionic liquid coating, an electrode, a mesh, and combinations thereof. In some embodiments, the matrix may comprise an ion-exchange resin, a polymeric resin and/or a water-wet protein attached to a hydrophilic surface, being a surface that is capable of forming an ionic or hydrogen bond with water and has a water contact angle of less than 90 degrees. For example, one or more NBPs may be present on a matrix with a thin layer of water-wet protein, which may maintain structure and function of the NBP. In some embodiments, the particles and/or beads may comprise a material selected from the group consisting of glass, ceramic, and a polymer (e.g., polyvinyl alcohol beads). In some embodiments, one or more NBPs and/or compositions may be dispersed into heated and melted ionic liquids, and following cooling, the one or more NBPs and/or compositions may be coated in an ionic liquid, which may improve stability of an NBP, for example, when contacted with organic solvents.

Additionally or alternatively, suitable carriers or scaffolds can comprise at least one transmembrane domain (e.g., alpha helical domain including hydrophobic residues, which can lock an NBP within a matrix), at least one peripheral membrane domain (e.g., signal proteins), and combinations thereof along with the one or more NBPs. In other embodiments, the NBPs can be semi-immobilized in a packed bed of a reactor.

### VI. Optional Method Steps

Additionally or alternatively, the methods can further comprise selecting one or more NBPs for contacting with the hydrocarbon stream based upon impurity type and content of the hydrocarbon stream. For example, the hydrocarbon stream may be tested to determine impurities content (e.g., nitrogen, sulfur, nickel and vanadium content) and properties. Then an NBP or mixture of NBPs may be selected based on the impurities present in the hydrocarbon stream and properties of the hydrocarbon stream. The NBP or mixture of NBPs may then be obtained or produced via methods known in the art, for example, the NBP(s) may be produced in Escherichia coli, the cells may be used whole or lysed, and the soluble fraction may be removed.

In other embodiments, methods of enhanced oil recovery using one or more NBP as described herein are provided. For example, one or more NBP, singularly or in combination with an injection fluid, may be introduced to an oil reservoir/wellbore. In some embodiments, the one or more NBP may reduce the viscosity of the oil present in the reservoir/wellbore allowing for increased oil recovery.

It is also contemplated herein that the NBPs described herein may be used in further refining processes, for example, the NBPs may be present in reactors for hydroprocessing, hydrofinishing, hydrotreating, hydrocracking, catalytic dewaxing (such as hydrodewaxing), solvent dewaxing, and combinations thereof.

### Examples

Without further description, it is believed that one of ordinary skill in the art can, using the preceding description and the following illustrative examples, make and utilize the NBPs and compositions described herein and practice the methods disclosed herein.

To test the ideas discussed above, eight NBPs were selected (Table 1) to screen for activity against aqueous soluble model nickel- and vanadium-containing porphyrins.

TABLE 1
Nickel-Binding Protein Properties

| | HypA | Hyp B | | Sly D |
|---|---|---|---|---|
| | Organism | | | |
| | H. pylori | E. coli | B. japonicum | E. coli |
| | Monomer size (kDa) | | | |
| | 25 | 27 | 33 | 21 |
| | Class | | | |
| | Chaperone | | | |
| | SEQ ID NO | | | |
| | 1 | 2 | 3 | 4 |
| Structure | Homodimer or | Homodimer or heterodimer with | | Heterodimer with |
| | heterodimer with | HypA or SlyD | | HypB |
| | HypB or UreE | | | |
| Ni-binding | 2 Ni (II) ions per | 2 Ni (II) ions per | 9 Ni (II) on 24- | C-terminal metal- |
| | dimer; N- | monomer. The | His rich N- | binding domain |
| | terminal | N-terminal Ni | terminus. | (MBD) has 28 (15 |
| | coordination by | binding domain | | His, 6 Cys, 7 |
| | His2, Glu3, & | has micromolar | | Asp/Glu) potential |
| | Asp40 | Kd, while the | | metal binding |
| | | SEQ ID NO: 12 | | residues with |
| | | binding sites in | | nanomolar Kd. |
| | | the C-terminal | | |
| | | domain have a | | |
| | | subpicomolar | | |
| | | Kd. | | |
| Additional metals | Zn (II) | Zn (II) | Zn (II) at dimer | First row transition |
| | | | interface; Zn (II), | metals |
| | | | Cu ((II), Co (II), | |
| | | | Cd (II), & Mn | |
| | | | (II) on N- | |
| | | | terminus. | |
| | UreE | GloA | NikR | Hpn |
| | Organism | | | |
| | E. aerogenes | E. coli | H. pylori | H. pylori |
| | Monomer size (kDa) | | | |
| | 17.5 | 15 | 17 | 7 |
| | Class | | | |
| | Chaperone | Metalloenzyme | Metalloregulator | Storage Protein |
| | SEQ ID NO | | | |
| | 5 | 6 | 7 | 8 |
| Structure | Homodimer or | Homodimer | | Monomer to 20- |
| | heterodimer with | | | mer complexes |
| | HypA | | | |
| Ni-binding | 6 Ni (II) per | 2 Ni (II), | 4 Ni (II) per | 5 Ni (II) per |
| | dimer | coordination by | complex at | monomer |
| | | His2 and Glu2 | nanomolar Kd | |
| | | | coordinated by | |
| | | | His88, His99, | |
| | | | His101, & Cys | |
| | | | 107; up to 10 Ni | |
| | | | (II) at | |
| | | | micromolar Kd | |
| Additional metals | Cd (II), Co (II), | Co (II), Cd (II) | Co (II), Cu (II), | Cu (II), Zn (II) |
| | Zn (II), Cu (II) | | Zn (II) | |

Three nickel- and one vanadium-containing model porphyrins (Ni(II)-protoporphyrin (IX); Ni(II)-meso-tetra-(4-carboxyphenyl) porphine; Ni(II)-meso-tetra-(N-methyl-4-pyridyl) porphine tetrachloride; & VO-meso-tetra-(N-methyl-4-pyridyl) porphine tetrachloride) were chosen as test substrates for the eight representative NBPs. These substrates were chosen because they are soluble in water, stable at concentrations compatible with UV-VIS spectroscopy, possess unique R-groups, and contain a nickel or vanadium center metal ion. The proteins' nickel- and vanadium-binding capabilities were assayed in a single phase, aqueous environment to eliminate mass transfer limitations that occur between multiple phases and stressful non-aqueous to conditions that may change protein structure and/or inhibit function. This approach minimizes false-negative results to maximize the number of potential protein candidates that can be identified.

UV-VIS spectroscopy was used for initial screening of protein activity against numerous model compounds because each model porphyrin has a characteristic UV-VIS absorbance spectrum. A shift in the wavelength of maximal absorbance, commonly referred to as the “Soret band,” indicates a change in porphyrin conformation or a protein-porphyrin interaction. A decrease in absorbance—or in the magnitude of the Soret band—implicates a change in intact porphyrin concentration, potentially caused by porphyrin demetallation.

Materials:

All nickel-binding protein expression strains are BL21-DE3(T1) E. coli and pET28b plasmid based. Expression plasmids were constructed by inserting the gene for each protein into the base vector pET28b. Each gene was codon-optimized for expression in E. coli and synthesized by ThermoFisher®. Restriction sites HindIII and NotI were used, on the 5′ and 3′ ends of each gene to digest and insert them in-frame relative to a 5′ HIS-tag. Antarctic phosphatase pre-treatment, 3:1 gene-insert:plasmid-backbone ratio, and Quick DNA Ligase were used for all ligation reactions. Ligation products were subcloned into chemically competent Top10 E. coli and confirmed by sequencing through Genewiz using forward (SEQ ID NO:9) and reverse (SEQ ID NO:10) primers. Plasmids from positive clones were transformed into the chemically competent BL21-DE3(T1) E. coli expression strain. Plasmid extractions were done using QIAprep® spin Miniprep® kits and DNA purifications using QIAquick® PCR purification kits. All cloning and expression strains were stored at −80° C. in 25% glycerol stocks.

Liquid LB and LB-agar plates were used for culture growth. 100 μg/mL ampicillin (AMP100) or 50 μg/mL kanamycin (KAN50) was used for selection with strains harboring nickel-binding protein encoding ThermoFisher® plasmids or pET28b-based protein expression vectors (respectively).

For expression of each plasmid, 5 mL of liquid LB-KAN50 was inoculated from a 25% glycerol −80° C. stock and grown overnight for 16 hrs. at 37° C. with shaking at 180 RPM. Non-baffled, 250 mL flasks containing 50 mL of LB-KAN50 were inoculated with 100 μL from overnight cultures (1:500 dilution) and grown at 37° C. and 180 rpm, to an optical density at 600 nm (OD₆₀₀) of 0.4-0.6 for induction. Nickel-binding protein expression was induced with 0.1 mM IPTG for 20 hrs. at room temperature and 90 RPM. Expression cultures were terminated by pelleting cells at 6 kRPM and 4° C. for 10 min. in an Avanti® J Series Centrifuge. Cell pellets were washed with phosphate-buffered saline (PBS), repelleted, supernatant decanted, and stored at −80° C. Cell pellets were resuspended in 5 mL protein storage buffer (PSB) and lysed with 5×30 sec. pulses (with 30 sec. rest between each) at 75% intensity on ice. Cell debris was pelleted at 10 kRPM and 4° C. for 30 min. and protein-containing supernatants transferred to chilled tubes in 1.5 mL aliquots. Protein lysates were stored at −80° C. for up to 3 months.

TABLE 2
Buffer compositions

| Solution | Ingredient | Concentration | |
|---|---|---|---|
| Phosphate buffered saline | NaHPO₄ | 80 | mM |
| | NaCl | 1.5M | |
| | KH₂PO₄ | 20 | mM |
| | KCl | 30 | mM |
| Protein storage buffer (pH 7.5) | Tris | 25 | mM |
| | EDTA | 1 | mM |
| | DTT | 5 | mM |
| | Glycerol | 20% | |
| Protein assay buffer | Tris | 25 | mM |
| | EDTA | 100 | μM |
| | DTT | 500 | μM |
| Porphyrin phosphate buffer | NaPO₄-monobasic | 5 | mM |
| (pH 7.0) | NaPO₄-dibasic | 5 | mM |
| | NaCl | 185 | mM |
| | Na₂EDTA | 1 | mM |
| Tris buffer (pH 7.8) | Tris | 20 | mM |
| | NaCl | 500 | mM |

To prepare crude protein, aliquots were thawed on ice and diluted 4-fold with protein assay buffer (PAB). To isolate purified nickel-binding proteins, protein aliquots were thawed on ice and proteins of interest were purified by HIS-tag-Ni-NTA column affinity followed by size exclusion chromatography with PD-10 desalting columns. Purified protein was eluted in PAB, collected in the first 2 mL PD-10 column flow-through fraction. Relative to the initial protein concentration, samples were diluted 4-fold to parallel crude protein samples.

Crude and purified protein quality was confirmed by gel separation and staining. Proteins were separated by size on a Bolt 4-12% Bis-Tris Plus gel in MES SDS running buffer, for 30 min. at 165 V and room temperature, stained with GelCode Blue Safe® Protein Stain for 1 hr. at 50 RPM and destained with water overnight at 50 RPM.

Model porphyrin stocks were prepared in porphyrin phosphate buffer (PPB), stored at 4° C. in amber bottles to protect samples from light, and used or discarded within one month.

Assay Conditions:

Amber tubes were used for all assays to protect samples from light. Crude and purified protein samples were combined with porphyrin substrates, to achieve final concentrations in Table 3, and total assay volumes were raised to 10 mL with Tris buffer. Absorbance of a 2.4 mL sample was measured in a 1 cm quartz cuvette, at room temperature, 1 nm resolution, and 0.7 sec. integration time on an Evolution201® UV-VIS spectrophotometer with Thermo INSIGHT® software. Samples were incubated for 24 hrs. at room temperature and 75 RPM and their final UV-VIS absorbance spectra was measured. An absorbance baseline was collected using Tris buffer at the beginning of each time point and 1% error was observed between technical replicates. Crude, empty pET28b vector (EV) strain lysate protein and PAB only controls were run in parallel with experimental samples (EXP) for each porphyrin.

TABLE 3
Porphyrin properties and details

| | Stock | Assay | |
|---|---|---|---|
| | Conc. | Conc. | Soret λ |
| Porphyrin | (mM) | (μM) | (nm) |
| Ni(II)-protoporphyrin (IX) | 100 | 50 | 400 |
| Ni(II)-meso-tetra(4-carboxyphenyl) | 50 | 10 | 400 |
| porphine | | | |
| Ni(II)-meso-tetra(N-methyl-4-pyridyl) | 50 | 10 | 440 |
| porphine tetrachloride | | | |
| VO-meso-tetra(N-methyl-4-pyridyl) | 50 | 10 | 422 |
| porphine tetrachloride | | | |

Porphyrin Demetallation Calculation was calculated according to the formula (EV_{f}−EXP_{f})÷EVₒ=% specific demetallation.

Chaperones:

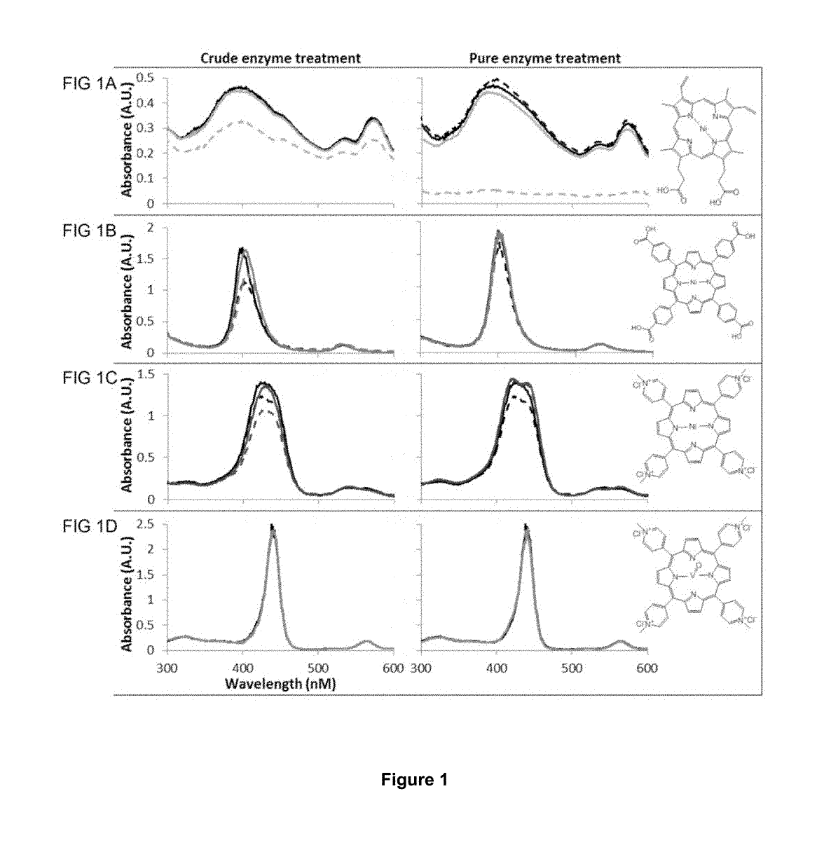

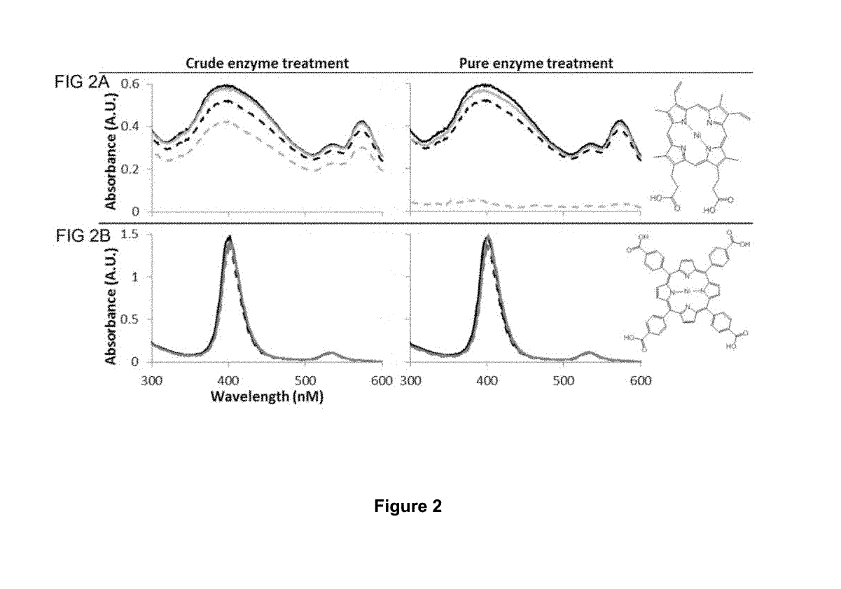

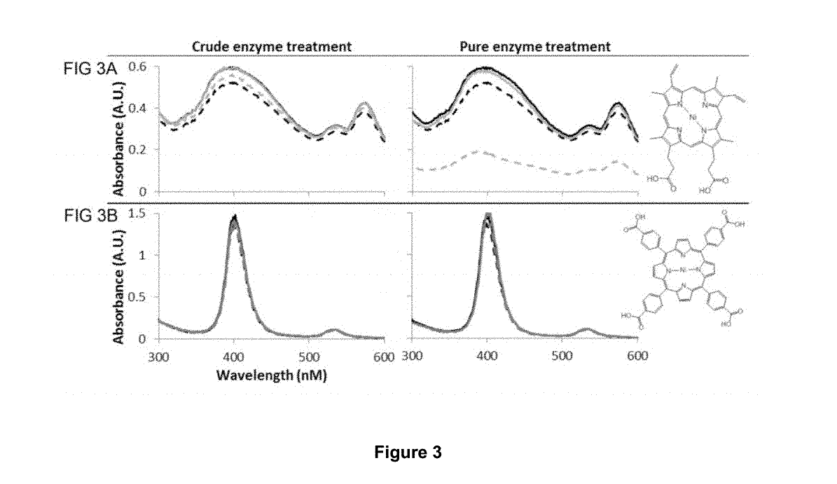

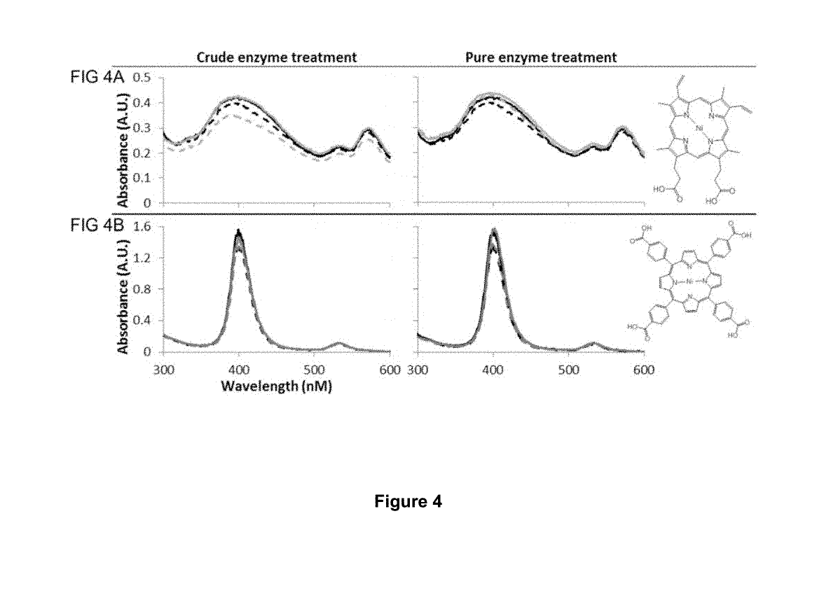

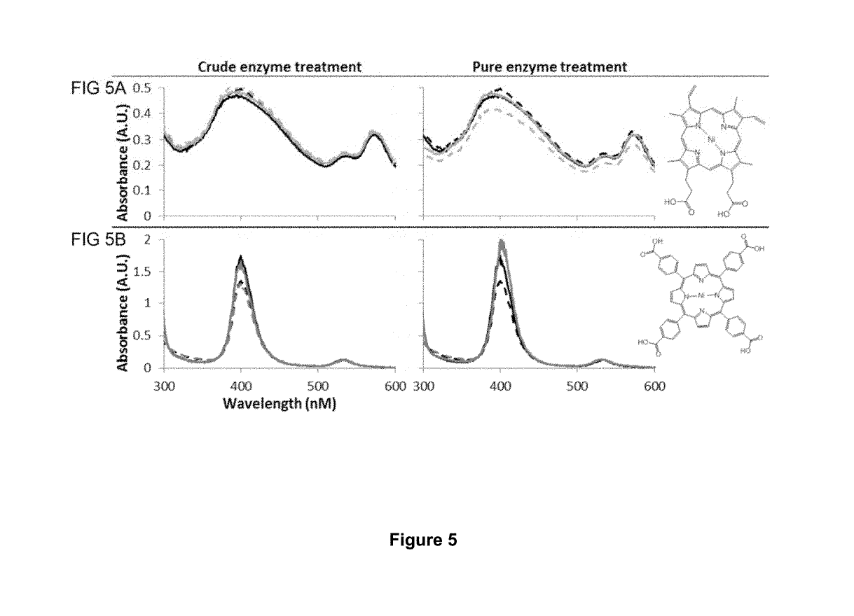

Model porphyrin binding and demetallation were assessed for five nickel chaperone proteins: HypB from B. japonicum (FIG. 1) and E. coli. (FIG. 2), HypA from H. pylori (FIG. 3), SlyD from E. coli (FIG. 4), and UreE from E. aerogenes (FIG. 5). Lysates and purification products from cultures transfected with an empty pET28b vector were used as a control.

Very similar activity was observed from both HypB species. Both HypB proteins displayed modest Ni(II)-protoporphyrin (IX) binding and demetallation capabilities as a component of crude protein lysate and significant activity upon purification. Crude lysates from B. japonicum reduced the Ni(II)-protoporphyrin (IX) Soret band by 29%, while purified B. japonicum HypB reduced the Soret band by 99% (FIG. 1A). Similarly, crude E. coli lysates reduced absorbance by 17% and purified E. coli HypB reduced absorbence by 83% (FIG. 2A). The tempered activity in crude lysate (relative to pure protein) may be due to non-specific interactions between components of the E. coli expression strain protein cocktail and the porphyrins and/or HypB. Non-specific protein-porphyrin interactions may protect the porphyrin from specific binding and demetallation by HypB. Alternatively, non-specific protein-HypB binding may prevent specific HypB-porphyrin binding and demetallation.

HypB activity was also tested against Ni(II)-meso-tetra(4-carboxyphenyl). Neither binding nor demetallation was observed with HypB from either B. japonicum (FIG. 1B) or E. coli (FIG. 2B). Ni(II)-meso-tetra(4-carboxyphenyl)'s R-group size and character are likely incompatible with these proteins' nickel-binding sites.

HypB from B. japonicum was also tested against Ni(II)- (FIG. 1C) and VO-meso-tetra(N-methyl-4-pyridyl) porphine tetrachloride (FIG. 1D). Neither binding to nor demetallation of these substrates was observed. The protein storage buffer was observed to induce a change to Ni(II)-mesotetra(N-methyl-4-pyridyl) porphine tetrachloride, indicated by the change in Soret band shape. Porphyrin reduction by DTT, or nickel chelation by EDTA are the two most likely causes of this change. As a result of the uncertain stability of these porphyrins in storage, these two were dropped from the screening profile and no additional NPBs were tested against these two porphyrins.

HypA did not display Ni(II)-protoporphyrin (IX) binding or demetallation capabilities as a component of crude protein lysate. However, significant activity was observed upon purification as Ni(II)-protoporphyrin (IX)'s Soret band was reduced by 59% (FIG. 3A). As discussed for HypB, the tempered activity in crude lysate (relative to pure protein) may be due to inhibitory, non-specific interactions between components of the E. coli expression strain protein cocktail and the porphyrins and/or HypA. After Ni(II)-protoporphyrin (IX), HypA activity was tested against Ni(II)-meso-tetra(4-carboxyphenyl). Similar to HypB, neither binding nor demetallation was observed upon treatment with HypA (FIG. 3B). Ni(II)-meso-tetra(4-carboxyphenyl)'s R-group size and character are likely incompatible with the protein's nickel-to binding sites.

Treatment of Ni(II)-protoporphyrin (IX) with crude SlyD lysates reduced the Soret band by 12%, but no change was observed from treatment with purified SlyD (FIG. 4A). This observation is consistent with the hypothesis that the purification process or post-purification conditions are impacting properties of the protein critical to acting on the nickel-containing substrate. Even modest changes to the protein's conformation may disrupt interactions with the porphyrin's nickel center. SlyD activity was tested against Ni(II)-meso-tetra(4-carboxyphenyl), but only minimal changes to the Soret band were observed (FIG. 4B).

Ni(II)-protoporphyrin (IX)'s Soret band remained unchanged upon treatment with crude UreE lysate and was reduced by 17% with purified UreE (FIG. 5A). Similar to HypA & HypB, the tempered activity on Ni(II)-protoporphyrin (IX) by UreE in crude lysate may be due to non-specific protein-porphyrin interactions that protect the porphyrin and non-specific protein-UreE binding that prevents UreE-porphyrin binding and demetallation. Neither binding nor demetallation of Ni(II)-meso-tetra(4-carboxyphenyl) was observed from UreE treatment (FIG. 5B).

Metalloregulators:

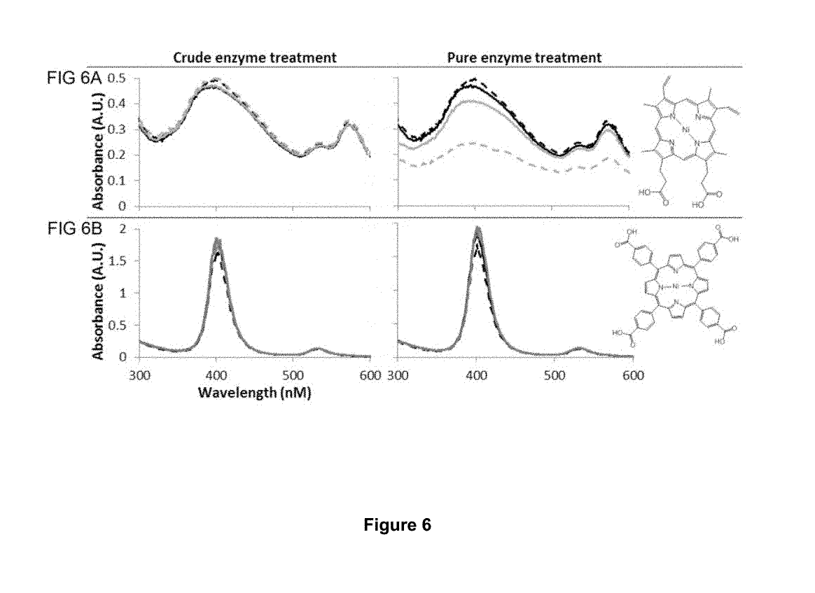

Binding and demetallation of Ni(II)-protoporphyrin (IX) and Ni(II)-meso-tetra(4-carboxyphenyl) by NikR from H. pylori were assessed by UV-VIS spectroscopy (FIG. 6). Ni(II)-protoporphyrin (IX)'s Soret band remained unchanged upon treatment with crude protein lysate and was reduced by 56% with purified NikR (FIG. 6A). Neither binding to nor demetallation was observed after NikR treatment of Ni(II)-protoporphyrin (IX) (FIG. 6B), suggesting that the R-group size and character of this substrate is likely incompatible with NikR's nickel-binding sites.

Metalloenzymes:

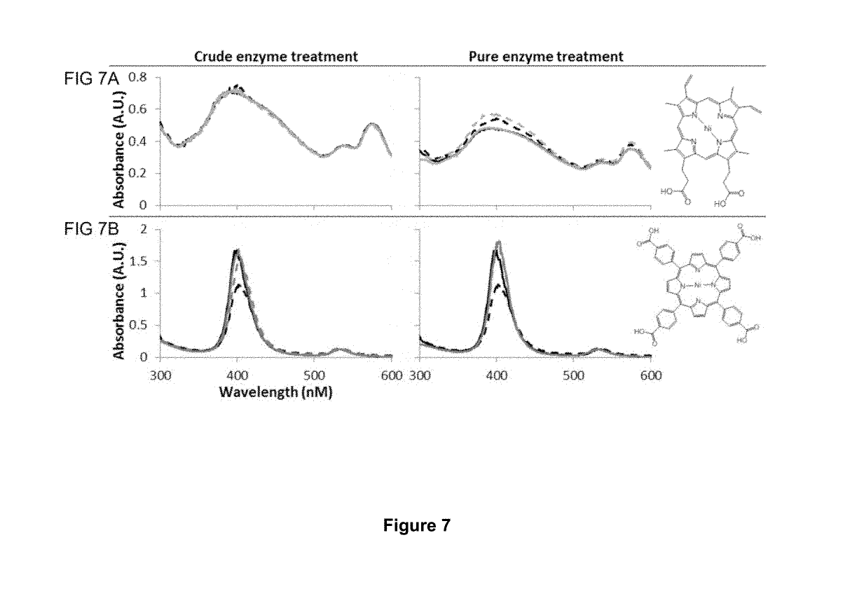

Binding and demetallation of Ni(II)-protoporphyrin (IX) (FIG. 7A) and Ni(II)-meso-tetra(4-carboxyphenyl) (FIG. 7B) by GloA from E. coli were assessed by UV-VIS spectroscopy. Neither GloA crude lysates nor purified GloA protein affected the Soret band for either model porphyrins, suggesting little or no binding and demetallation by GloA.

Nickel Storage Proteins:

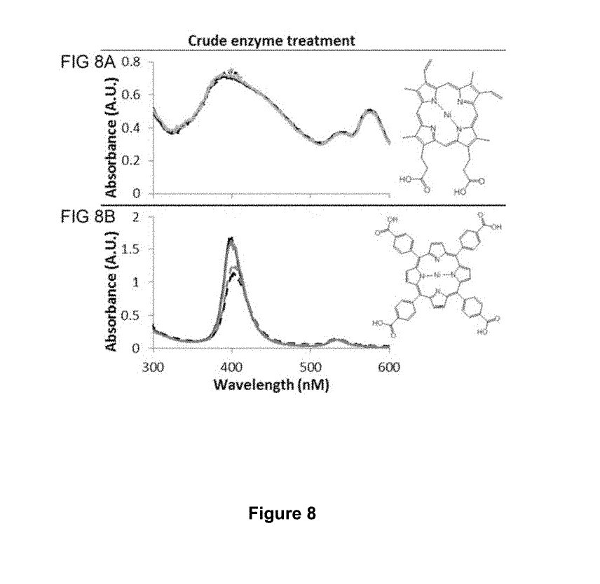

Binding and demetallation of Ni(II)-protoporphyrin (IX) (FIG. 8A) and Ni(II)-meso-tetra(4-carboxyphenyl) (FIG. 8B) by Hpn from H. pylori were assessed by UV-VIS spectroscopy. Hpn treatment did not affect the Soret band for either model porphyrin. Purification difficulties with Hpn prevented the testing of pure Hpn against the model compounds.

The characteristic Soret band shift that often results from protein-porphyrin interactions was not observed with these porphyrins upon treatment with any of the NBPs in Table 1, either as a component of a crude protein lysate or purified protein. Naturally occurring porphyrin-binding proteins contain an active site in which the porphyrin binds for demetallation. Porphyrin reduction and/or a planar to non-planar conformational change (porphyrin “ruffling”) are typically induced upon porphyrin binding in the active site, and research shows that both of these changes can alter porphyrins' absorbance characteristics. In contrast, the eight NPBs in Table 1 do not possess active sites. Without being bound by theory, it is possible that the Table 1 NBPs predominantly interact with the porphyrins' center metal ion via short nickelbinding sequence and minimally with the tetrapyrrole ring. Demetallation may then proceed without generating a reduced porphyrin intermediate or distorting the planar tetrapyrrole ring. Additionally, slow nickel-binding protein-porphyrin binding may be followed by rapid demetallation, minimizing the amount of protein-bound, reduced or ruffled, porphyrin present. Thus, it is reasonable that binding and demetallation of the model porphyrins tested may occur without reduction, ruffling, or a shift in the Soret band.

TABLE 4
Sequence Correspondence Table

| SEQ ID NO | Protein | Organism |
|---|---|---|
| 1 | HypA | Bradyrhizobium japonicum |
| 2 | HypB | Escherichia coli |
| 3 | HypB | Helicobacter pylori |
| 4 | SlyD | Escherichia coli |
| 5 | UreE | Enterobacter aerogenes |
| 6 | GloA | Escherichia coli |
| 7 | NikR | Helicobacter pylori |
| 8 | Hpn | Helicobacter pylori |

| | |
|---|---|
| | Construct |
| 9 | Forward cloning primer |
| 10 | Reverse cloning primer |
| 11 | Peptide motif from Hpn |
| 12 | Peptide motif from E. coli HypB |

1. A method of biologically upgrading a hydrocarbon stream comprising contacting the hydrocarbon stream with a nickel-binding protein (NBP).

2. The method of claim 2, wherein the nickel-binding protein is substantially cell-free.

3. The method of claim 1, wherein the nickel-binding protein is a recombinant protein.

4. The method of claim 1, wherein the nickel-binding protein classifies as belonging to at least one Pfam family selected from the group consisting of PF01155, PF02492, PF00254, PF05194, PF00903, and PF08753.

5. The method of claim 1, wherein the nickel-binding protein has at least 85% sequence identity to a polypeptide selected from the group consisting of SEQ ID NOs: 1-8.

6. The method of claim 1, wherein the biological upgrading comprises removing impurities from the hydrocarbon.

7. The method of claim 6, wherein the impurities comprise nickel, cadmium, vanadium, or arsenic.

8. The method of claim 7, wherein the metal is nickel or vanadium.

9. The method of claim 1, wherein the hydrocarbon stream is crude oil or vacuum residual.

10. The method of claim 1, wherein the contacting is performed at a temperature from about 15° C. to about 90° C.

11. The method of claim 1, wherein the nickel-binding protein is thermally stable from about 90° C. to about 120° C.

12. The method of claim 1, further comprising selecting one or more nickel-binding protein for the contacting step based upon impurity type and content in the hydrocarbon stream.

13. The method of claim 1, wherein there is less than 10% (wt/wt) loss of hydrocarbon following separating the impurities from the hydrocarbon stream.

14. The method of claim 1, further comprising contacting the hydrocarbon stream with an oxygenase, a NBP and/or another NBP.

15. The method of claim 1, wherein the nickel-binding protein is present in an oil reservoir, a pipeline, a tank, a vessel, a reactor, and/or a waste water stream.

16. The method of claim 1, wherein the nickel-binding protein is in free form, crystal form, and/or immobilized on a carrier.

17. The method of claim 16, wherein the carrier is selected from the group consisting of a membrane, a filter, a matrix, diatomaceous material, particles, beads, an ionic liquid, an electrode, a mesh, and a combination thereof.

18. The method of claim 17, wherein the matrix comprises an ion-exchange resin, a polymeric resin and/or a water wet protein.

19. The method of claim 17, wherein the particles and/or beads comprise a material selected from the group consisting of glass, ceramic, and a polymer.

20. The method of claim 1, wherein the nickel-binding protein is hydrophobically modified to be at least 10% more enriched in hydrophobic amino acids selected from the group consisting of Ala, Gly, Ile, Leu, Met, Pro, Phe, and Trp.

21. The method of claim 20, wherein the nickel-binding protein is selected from the group consisting of SEQ ID NOs: 1-8.

22. The method of claim 20, wherein the enrichment is at least 20%.

23. The method of any one of claim 20, wherein enrichment is achieved by replacing a native residue with a hydrophobic amino acid.

24. The method of claim 20, wherein enrichment is achieved by adding a hydrophobic amino acid between two native residues.

25. The method of claim 1, wherein the nickel-binding protein is rinsed with n-propanol.

26. The method of claim 1, wherein the nickel-binding protein is conjugated to a polyethylene glycol.

27. The method of claim 1, wherein disulfide bridges are added to the nickel-binding protein.

28. The method of claim 1, wherein one to ten hydrophobic amino acids are added to an amino or carboxy terminus of the nickel-binding protein, wherein the hydrophobic amino acid is selected from the group consisting of Ala, Gly, Ile, Leu, Met, Pro, Phe, and Trp.

29. A recombinant polypeptide having at least 70% sequence identity but no more than 90% sequence identity to any one of SEQ ID NOs: 1-8, wherein the sequence is manipulated to be at least 10% more enriched in hydrophobic amino acids relative to the sequence selected from SEQ ID NOs: 1-8, and wherein the hydrophobic amino acids are selected from the group consisting of Ala, Gly, Ile, Leu, Met, Pro, Phe, and Trp.

30. The recombinant polypeptide of claim 29, wherein the enrichment is at least 20%.

31. An isolated or recombinant nucleic acid molecule comprising a sequence encoding the polypeptide of claim 29.

32. A vector comprising the nucleic acid molecule of claim 31.
